(19) United States
(12) Patent Application Publication
(10) Pub. No.: US 2012/0083857 A1
(43) Pub. Date: Apr. 5, 2012

(54) TISSUE STIMULATION SYSTEM AND METHOD WITH ANATOMY AND PHYSIOLOGY DRIVEN PROGRAMMING

(75) Inventors: Kerry Bradley, Glendale, CA (US); Bradley Hershey, Valencia, CA (US)

(73) Assignee: BOSTON SCIENTIFIC NEUROMODULATION CORPORATION, Valencia, CA (US)

(21) Appl. No.: 13/244,435

(22) Filed: Sep. 24, 2011

Related U.S. Application Data
(60) Provisional application No. 61/390,112, filed on Oct. 5, 2010.

(51) Int. Cl. A61N 1/372

(52) U.S. Cl. 607/46

(57) ABSTRACT

An external control device for use with a tissue stimulation device and at least one tissue stimulation lead having a plurality of electrodes implanted within a patient comprises a user interface configured for allowing a user to enter first information defining a therapeutic indication and second information defining the location of the at least one tissue stimulation lead relative to an anatomical reference, at least one processor configured for analyzing the first and second information and generating a set of stimulation parameters based on the analysis, and output circuitry configured for transmitting the at least one stimulation parameter set to the tissue stimulation device.

### RELATED APPLICATION DATA

The present application claims the benefit under 35 U.S.C. §119 to U.S. provisional patent application Ser. No. 61/390,112, filed Oct. 5, 2010. The foregoing application is hereby incorporated by reference into the present application in its entirety.

### FIELD OF THE INVENTION

The present inventions relate to tissue stimulation systems, and more particularly, to tissue stimulation systems for programming tissue stimulation leads.

### BACKGROUND OF THE INVENTION

Implantable tissue stimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable tissue stimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The tissue stimulation system may further comprise an external control device to remotely instruct the neurostimulator to generate electrical stimulation pulses in accordance with selected stimulation parameters.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width, and rate of the electrical pulses provided through the electrode array. Each electrode combination, along with the electrical pulse parameters, can be referred to as a “stimulation parameter set.”

With some tissue stimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of pain), while minimizing the volume of non-target tissue that is stimulated.

However, the number of electrodes available, combined with the ability to generate a variety of complex stimulation pulses, presents a huge selection of stimulation parameter sets to the clinician or patient. For example, if the tissue stimulation system to be programmed has an array of sixteen electrodes, millions of stimulation parameter sets may be available for programming into the tissue stimulation system. Today, tissue stimulation system may have up to thirty-two electrodes, thereby exponentially increasing the number of stimulation parameters sets available for programming.

To facilitate such selection, the clinician generally programs the neurostimulator through a computerized programming system. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets, which will typically be those that stimulate all of the target tissue in order to provide the therapeutic benefit, yet minimizes the volume of non-target tissue that is stimulated. The computerized programming system may be operated by a clinician attending the patient in several scenarios.

For example, in order to achieve an effective result from SCS, the lead or leads must be placed in a location, such that the electrical stimulation will cause paresthesia. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system. Thus, correct lead placement can mean the difference between effective and ineffective pain therapy. When electrical leads are implanted within the patient, the computerized programming system, in the context of an operating room (OR) mapping procedure, may be used to instruct the neurostimulator to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neurostimulator, with a set of stimulation parameters that best addresses the painful site. Thus, the navigation session may be used to pinpoint the stimulation region or areas correlating to the pain. Such programming ability is particularly advantageous for targeting the tissue during implantation, or after implantation should the leads gradually or unexpectedly move that would otherwise relocate the stimulation energy away from the target site. By reprogramming the neurostimulator (typically by independently varying the stimulation energy on the electrodes), the stimulation region can often be moved back to the effective pain site without having to re-operate on the patient in order to reposition the lead and its electrode array. When adjusting the stimulation region relative to the tissue, it is desirable to make small changes in the proportions of current, so that changes in the spatial recruitment of nerve fibers will be perceived by the patient as being smooth and continuous and to have incremental targeting capability.

One known computerized programming system for SCS is called the Bionic Navigator®, available from Boston Scientific Neuromodulation Corporation. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), may be stored in both the Bionic Navigator® and the remote control and combined into a stimulation program that can then be used to stimulate multiple regions within the patient.

While the use of the Bionic Navigator® has been particularly useful in programming tissue stimulation systems, targeting specific regions in the context of SCS can be challenging to the inexperienced user. For example, if the patient requires therapy for chronic pain in the foot, a physician or clinician who has not previously programmed a tissue stimulation system associated with a patient with foot pain may be unsure as to the set of stimulation parameters to begin with, and may therefore, require an extended amount of time to find an effective set of stimulation parameters or, in the worst case, may not find an effective set of stimulation parameters at all.

### SUMMARY OF THE INVENTION

In accordance with the present inventions, an external control device for use with a tissue stimulation device and at least one tissue stimulation lead having a plurality of electrodes implanted within a patient is provided.

The external control device comprises a user interface configured for allowing a user to enter first information defining a therapeutic indication (e.g., chronic pain) and second information defining the location of the tissue stimulation lead(s) relative to an anatomical reference (e.g., a vertebral level and/or mediolateral location relative to a spine) and optionally the type and number of the tissue stimulation lead(s), and if multiple tissue stimulation leads are used, the positional of the tissue stimulation leads relative to each other. The user interface may optionally be configured for allowing the user to optimize at least one stimulation parameter set.

The external control device further comprises processing circuitry configured for analyzing the first and second information and generating the stimulation parameter set(s) based on the analysis, and output circuitry (e.g., telemetry circuitry) configured for transmitting the set of stimulation parameters to the tissue stimulation device. The external control device may include a housing containing the user interface, processing circuitry, and output circuitry.

In one embodiment, the external control device further comprises memory storing a database containing a plurality of reference therapeutic indications (e.g., different pain regions) and a plurality of desired stimulation targets respectively corresponding to the reference therapeutic indications, which may be empirically determined. In this case, the processing circuitry is configured for analyzing the first information by comparing the defined therapeutic indication to the stored reference therapeutic indications and selecting at least one of the desired stimulation targets based on this comparison, for analyzing the second information by comparing the defined location of the tissue stimulation lead(s) to a location of the desired stimulation target(s) within the anatomical reference, and for selecting one or more of the electrodes adjacent the desired stimulation target(s) based on this comparison. The selected electrode(s) will then be included within the generated stimulation parameter set(s).

The database may further contain a plurality of pulsewidth values respectively corresponding to the reference therapeutic indications, in which case, the processing circuitry may be configured for selecting one of the pulsewidth values based on the comparison of the defined therapeutic indication to the stored reference therapeutic indications. The selecting pulse width value will then be included within the generated stimulation parameter set(s). The database may further contain a plurality of anode-cathode patterns respectively corresponding to the reference therapeutic indications, in which case, the processing circuitry may be configured for selecting an anode-cathode pattern based on the comparison of the defined therapeutic indication to the stored reference therapeutic indications, and selecting the electrodes based on the selected anode-cathode pattern.

In another embodiment, the processing circuitry is configured for analyzing the first information and second information using a heuristic set of rules.

For example, the generated stimulation parameter set(s) may comprise a pulse width, in which case, the heuristic set of rules may comprise selecting a relatively low pulse width value (e.g., in the range of 50-300 μs) as the pulse width if the defined therapeutic indication is of the type that is treated via stimulation of dorsal roots and/or large nerve fibers, and selecting a relatively high pulse width value (e.g., in the range of 300-2000 μs) as the pulse width if the defined therapeutic indication is of the type that is treated via stimulation of dorsal columns and/or small nerve fibers.

As another example, the generated stimulation parameter set(s) may comprise an active electrode combination, in which case, the heuristic set of rules may comprise selecting a relatively narrow anode-cathode separation (e.g., less than 8 mm) for the active electrode combination if the defined therapeutic indication is of the type that is treated via stimulation of midline dorsal column nerve fibers, selecting a relatively medial anode-cathode separation (e.g., in the range of 8 to 24 mm) for the active electrode combination if the defined therapeutic indication is of the type that is treated via stimulation of lateral dorsal column nerve fibers, and selecting a relatively wide anode-cathode separation (e.g., greater than 24 mm) for the active electrode combination if the defined therapeutic indication is of the type that is treated via stimulation of dorsal root nerve fibers.

As still another example, the generated stimulation parameter set(s) may comprise an active electrode combination, in which case, the set of heuristic rules may comprise selecting for the electrode combination a cathode over the midline dorsal column fibers if the active electrode combination is caudal to the dorsal root fibers innervating a tissue region associated with the defined therapeutic indication, and selecting for the electrode combination a cathode lateral to the midline dorsal column fibers if the active electrode combination is at the same level as the dorsal root fibers innervating the tissue region associated with the defined therapeutic indication.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a spinal cord stimulation (SCS) system constructed in accordance with one embodiment of the present inventions;

FIG. 2 is a perspective view of the arrangement of the SCS system of FIG. 1 with respect to a patient;

FIG. 3 is a profile view of an implantable pulse generator (IPG) and percutaneous leads used in the SCS system of FIG. 1;

FIG. 4 is front view of a remote control (RC) used in the SCS system of FIG. 1;

FIG. 5 is a block diagram of the internal components of the RC of FIG. 4;

FIG. 6 is a block diagram of the internal components of a clinician's programmer (CP) used in the SCS system of FIG. 1;

FIG. 7 is a patient profile screen that can be displayed by the CP of FIG. 6;

FIG. 8 is a lead configuration screen that can be displayed by the CP of FIG. 6;

FIG. 9 is a lead orientation screen that can be displayed by the CP of FIG. 6;

FIG. 10 is a drag-and-drop lead screen that can be displayed by the CP of FIG. 6; and

FIG. 11 is a flow diagram illustrating one of programming the IPG of FIG. 3 using the CP of FIG. 6.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neurostimulator configured to treat urinary incontinence, sleep apnea, shoulder subluxation, headache, etc.

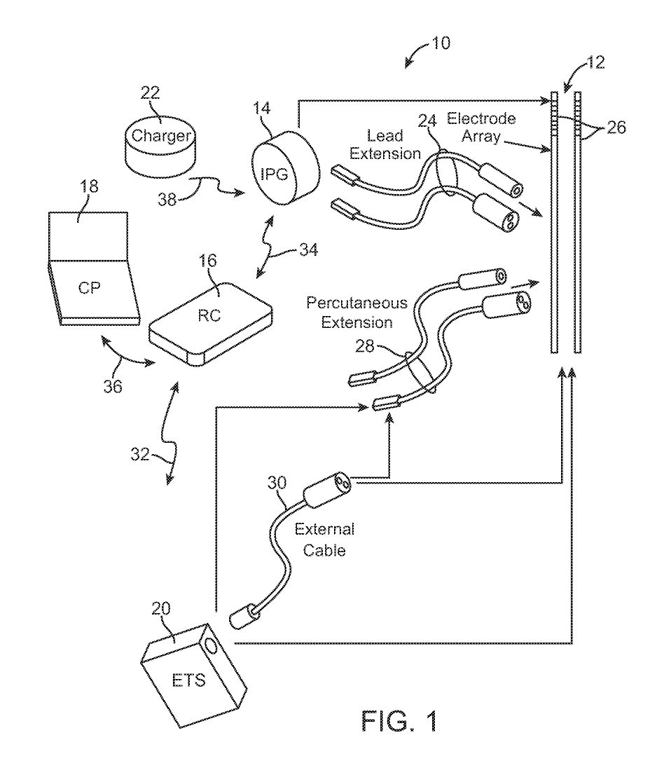

Turning first to FIG. 1, an exemplary SCS system 10 generally includes a plurality (in this case, two) of implantable tissue stimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the tissue stimulation leads 12, which carry a plurality of electrodes 26 arranged in an array. In the illustrated embodiment, the tissue stimulation leads 12 are percutaneous leads, and to this end, the electrodes 26 are arranged in-line along the tissue stimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the tissue stimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the tissue stimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and tissue stimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

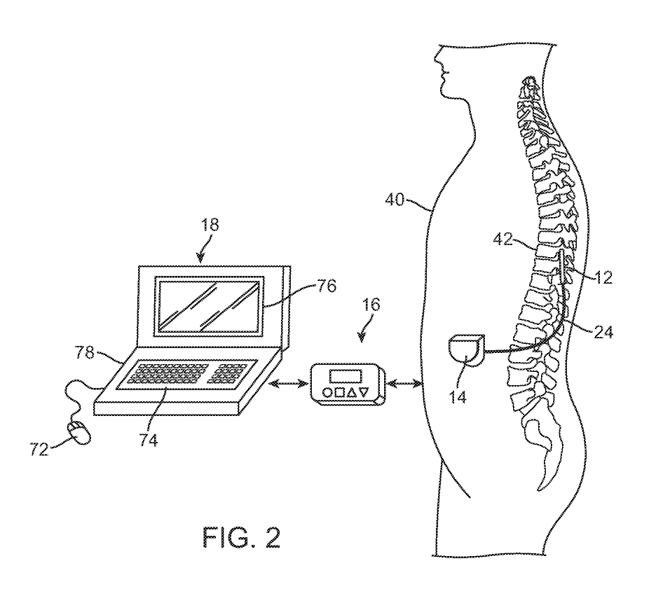

As shown in FIG. 2, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

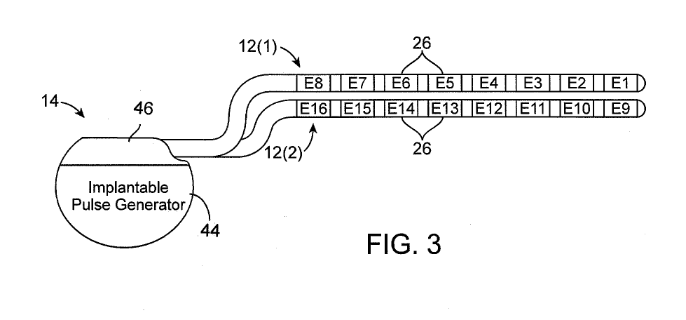

Referring now to FIG. 3, the external features of the tissue stimulation leads 12 and the IPG 14 will be briefly described. One of the tissue stimulation leads 12(1) has eight electrodes 26 (labeled E1-E8), and the other tissue stimulation lead 12(2) has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the tissue stimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode combinations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case 44 of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the tissue stimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

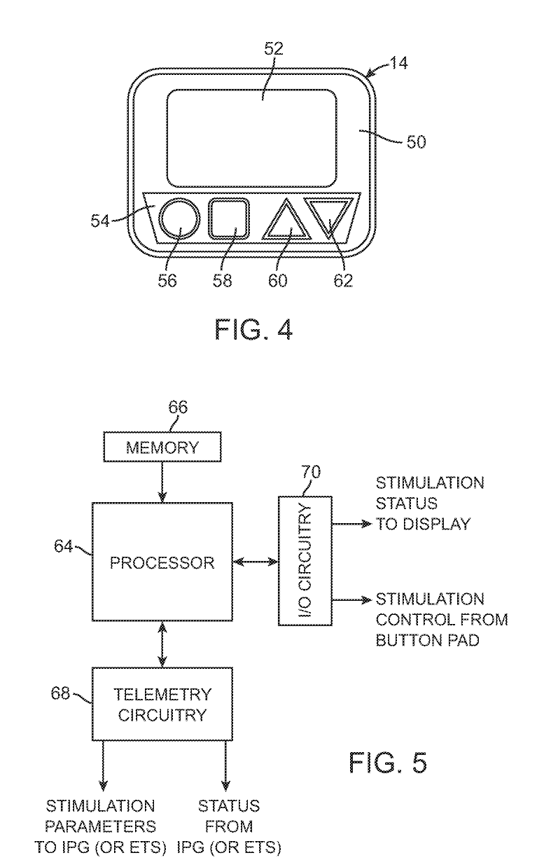

Referring now to FIG. 4, one exemplary embodiment of an RC 16 will now be described. As previously discussed, the RC 16 is capable of communicating with the IPG 14, CP 18, or ETS 20. The RC 16 comprises a casing 50, which houses internal componentry (including a printed circuit board (PCB)), and a lighted display screen 52 and button pad 54 carried by the exterior of the casing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touch screen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 (or ETS 20) via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implemented using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

To allow the user to perform these functions, the CP 18 includes a mouse 72, a keyboard 74, and a programming display screen 76 housed in a case 78. It is to be understood that in addition to, or in lieu of, the mouse 72, other directional programming devices may be used, such as a trackball, touchpad, joystick, or directional keys included as part of the keys associated with the keyboard 74. In alternative embodiment, the display screen 76 may take the form of a touch screen. The CP 18 further includes detection circuitry 80 capable of detecting an actuation event on the display screen 76. Such actuation event may include placing at least one pointing element (not shown) in proximity to at least one graphical object displayed on the display screen 76, as well as possibly other events involving the point element(s), such as moving the pointing element(s) across the screen or clicking or tapping with the pointing element(s), as will be described in further detail below.

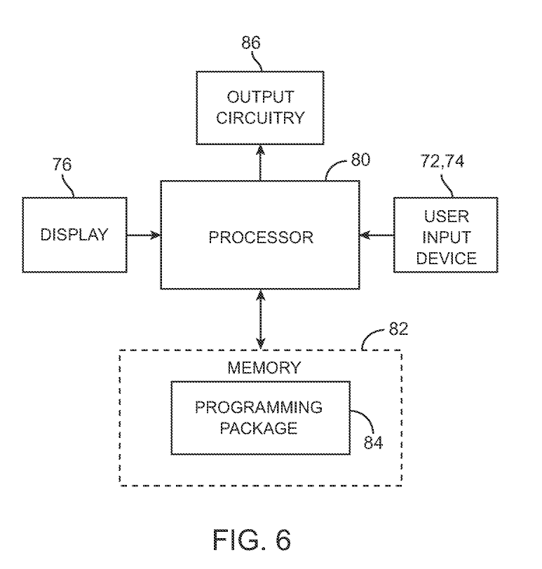

As shown in FIG. 6, the CP 18 generally includes a processor 82 (e.g., a central processor unit (CPU)) and memory 84 that stores a stimulation programming package 86, which can be executed by the processor 82 to allow the user to program the IPG 14, and RC 16. The CP 18 further includes output circuitry 88 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Execution of the programming package 86 by the processor 82 provides a multitude of display screens (not shown) that can be navigated through via use of the mouse 72. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the leads 12, and select and program the IPG 14 with stimulation parameters in both a surgical setting and a clinical setting. Further details discussing the above-described CP functions are disclosed in U.S. patent application Ser. No. 12/501,282, entitled “System and Method for Converting Tissue Stimulation Programs in a Format Usable by an Electrical Current Steering Navigator,” and U.S. patent application Ser. No. 12/614,942, entitled “System and Method for Determining Appropriate Steering Tables for Distributing Stimulation Energy Among Multiple Neurostimulation Electrodes,” which are expressly incorporated herein by reference.

Most pertinent to the present inventions, execution of the programming package 86 provides a user interface that allows the user to enter information defining a therapeutic indication of the patient (e.g., any of a plurality of different tissue regions associated with chronic pain) and information defining a location of the tissue stimulation lead or leads 12 relative to an anatomical reference (in this case, a vertebral location and/or mediolateral position relative to a physiological centerline of the spine of the patient), as well as additional information related to the tissue stimulation leads.

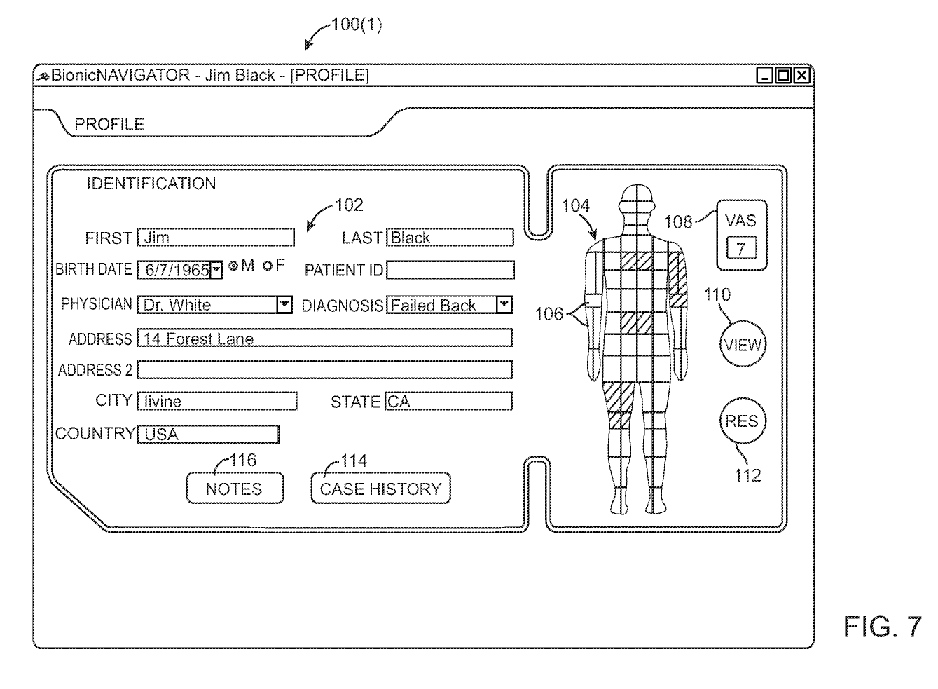

For example, as shown in FIG. 7, a patient profile screen 100(1) generated by the CP 18 includes a multitude of identification boxes 102 that allows the clinician to create, edit information required to generate or update a patient record, such as, e.g., name, birth date, patient identification, physician, diagnosis, and address. The patient profile screen 100(1) also provides a pain map of the human body 104 divided into several regions 106. Clicking on one or more of these regions 106 allows the clinician to record the regions of pain experienced by the patient. Alternatively, the pain region information may be entered verbally, through text entry, etc. In the illustrated embodiment, the upper back, lower back, right arm, and left thigh of the patient are highlighted, indicating that these are the regions of pain experienced by the patient. The patient profile screen 100(1) also has a visual analog scale (VAS) 108 that can be clicked to allow the clinician to manually record the amount of pain experienced by the patient from a scale of 0 (no pain) to 10 (worst imaginable pain), both without therapy and during therapy. The patient profile screen 100(1) also has a view button 110 that can be clicked to toggle the pain map 104 between a front view and a rear view, and a resolution button 112 that can be clicked to toggle the resolution of the regions 106 in the pain map 104 between low and high. The patient profile screen 100(1) also has a case history button 114 that can be clicked to allow the clinician to review the date and time of each procedure performed on the patient, and a notes button 116 that can be clicked to allow the clinician to enter notes in a free-form manner that can be subsequently reviewed in the case history.

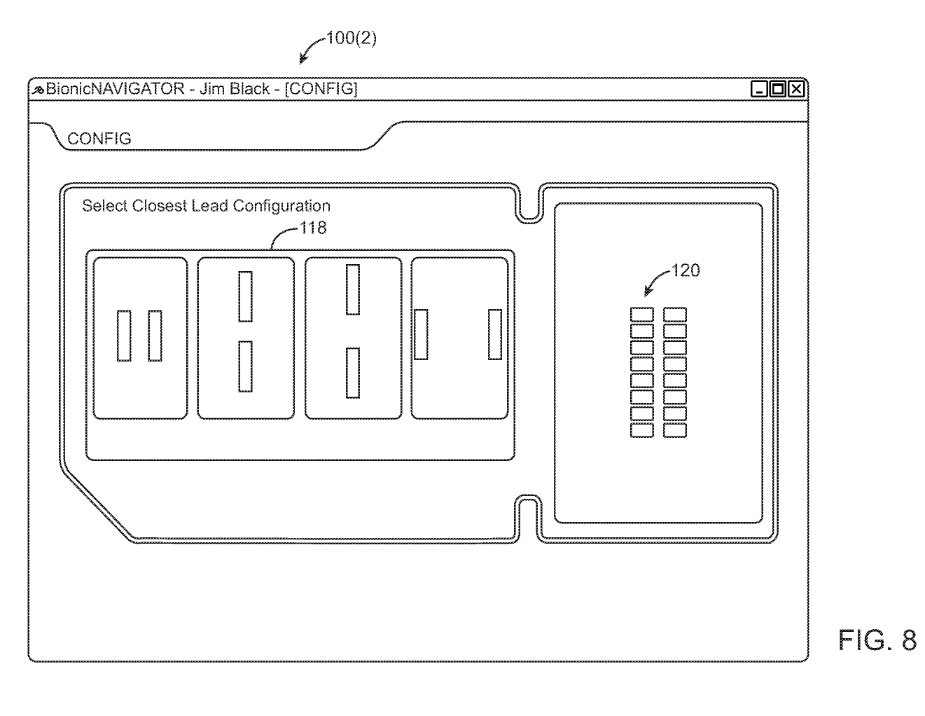

A lead type screen (not shown) generated by the CP 18 has a pull-down menu or list (not shown) that allows a user to enter a model type of lead and number of leads. In the conventional case where a pair of percutaneous leads are to be used, a lead configuration screen 100(2) generated by the CP 18 includes four different graphical configurations 118 that can be clicked on to select a specific lead configuration (e.g., a closely spaced side-by-side configuration, a closely spaced top-bottom configuration, a widely spaced top-bottom configuration, or a widely spaced side-by-side configuration) that best matches the actual configuration of the implanted leads 12, as shown in FIG. 8.

In this case, the closely spaced side-by-side configuration is shown selected, which is shown in a graphical representation of two electrode octets 120.

Alternatively, rather than inputting the lateral spacing between the leads 12 using the lead configuration screen 100(2), the positions of the tissue stimulation leads 12 relative to each other can be determined based on the measured electrical parameters in a conventional manner, such as, e.g., any one or more of the manner disclosed in U.S. Pat. No. 6,993,384, entitled “Apparatus and Method for Determining the Relative Position and Orientation of Tissue stimulation leads,” U.S. patent application Ser. No. 12/550,136, entitled “Method and Apparatus for Determining Relative Positioning Between Tissue stimulation leads,” and U.S. patent application Ser. No. 12/623,976, entitled “Method and Apparatus for Determining Relative Positioning Between Tissue stimulation leads,” which are expressly incorporated herein by reference.

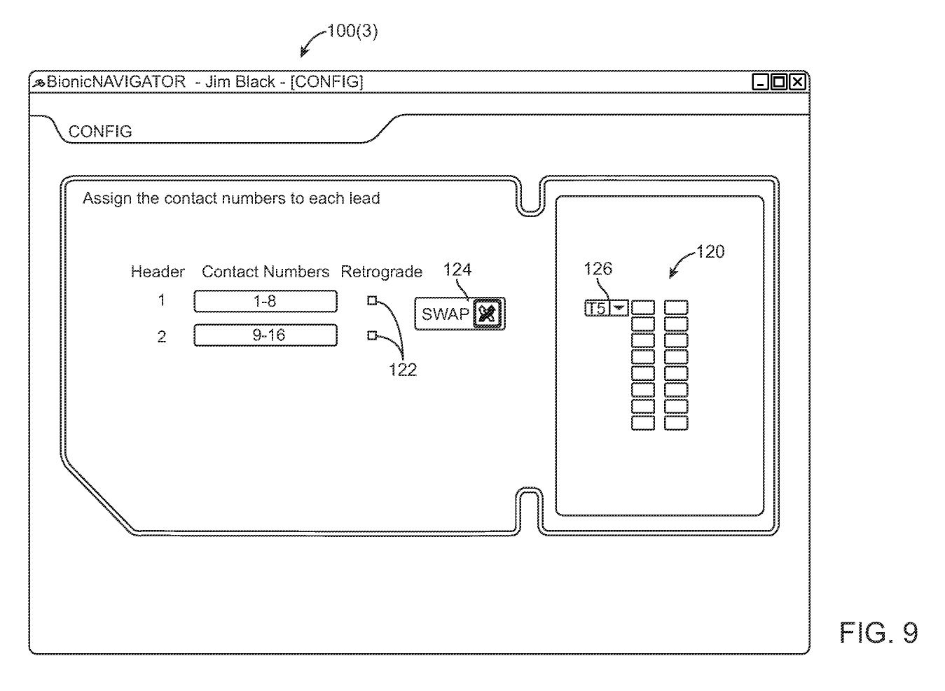

As shown in FIG. 9, a lead orientation screen 100(3) generated by the CP 18 allows the clinician to select the lead direction, assign the electrode numbers to each lead, and the vertebral position of the leads. In particular, the lead orientation screen 100(3) has a retrograde box 122 that can be clicked to indicate how the lead is vertically oriented. In this case, neither of the retrograde boxes 122 has been checked, so that first octet of electrodes will be numbered from 1 to 8 starting from the top of the first lead, and the second octet of electrodes will be numbered from 9 to 16 starting from the top of the second lead. However, in the case where the first retrograde box 122 is checked, the first octet of electrodes will be numbered from 8 to 1 starting from the top of the first lead, and the second octet of electrode will be numbered from 16 to 9 starting from the top of the second lead. The lead orientation screen 100(3) also has a swap button 124 that can be clicked to associate the electrode octets (1-8 and 9-16) to the first and second leads, with the nominal designation being electrodes 1-8 on the first lead and electrodes 9-16 on the second lead. The lead orientation screen 100(3) has a vertebral location pull down menu 126 next to the graphical electrode representation 120 that a clinician can use to indicate the vertebral position of the leads (e.g., C1-C7.5, T1-T12.5, L1-L5.5, S1-S5). In the example, the T5 vertebral position has been selected. The lead orientation screen 100(3) may further include a control (not shown) that allows the longitudinal stagger between the leads to be defined, e.g., by repeatedly clicking a button that incrementally moves the electrode octets relative to each other.

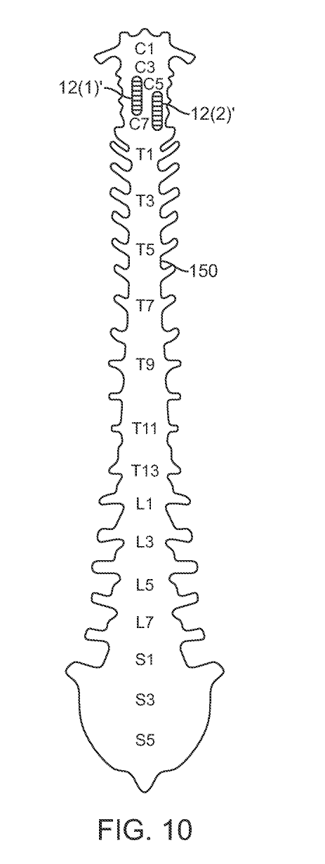

In an optional embodiment, one or more virtual leads 12′ can be dragged and dropped from a lead generation icon (not shown) over a graphical representation of an anatomical region 150 (in this case, a spine) at a location matching the location of the anatomical region at which the actual lead(s) 12 are implanted, as shown in a drag-and-drop lead screen 100(4) of FIG. 10. For example, one object can be dragged and dropped from the lead generation icon to generate one of the virtual leads 12(1)′, and another object can be dragged and dropped from the lead generation icon to generate another one of the virtual leads 12(2)′ relative to the previously generated virtual lead 12(1)′, such that the reference lead configuration can be user-defined. The longitudinal distance and/or lateral distance between the virtual leads 12′ can be defined in this manner. Each vertebra of the spine 150 is labeled with the corresponding level, so that the user can drop the respective virtual lead 12′ at the vertebra corresponding to the location of the actual lead 12 relative to the spine.

The manner in which a virtual lead is selected, dragged, and dropped will depend on the nature of the user interface. For example, if the display screen 76 is conventional, and a mouse 72 or other pointing device is used, the user may drag and drop the virtual leads 12′ onto the anatomical structure 150 using a cursor. If the display screen 76 is a digitizer screen, the user may drag and drop the virtual leads 12′ onto the anatomical structure 150 using a stylus or finger. Further details discussing the dragging and dropping of virtual leads 12′ onto a screen are provided in U.S. Provisional Application Ser. No. 61/333,673, entitled “System and Method for Defining Tissue stimulation lead Configurations,” which is expressly incorporated herein by reference.

Thus, it can be appreciated from the foregoing that the user interface of the CP 18 allows the user to enter information defining a therapeutic indication and information defining the type, number, and vertebral and mediolateral locations of the tissue stimulation leads 12. By analyzing this information, the CP 18 can automatically generate a set of stimulation parameters that serves as a starting point that is close or is as close as possible to the optimum set of stimulation parameters, thereby allowing the user to more efficiently program the IPG 14 (or ETS 20).

In one embodiment, the memory 84 stores a database containing a plurality of reference therapeutic indications (in this case, a plurality of pain regions) and a plurality of desired stimulation targets respectively corresponding to the therapeutic indications. For example, the pain regions and corresponding desired stimulation targets may be generated and stored in accordance with a conventional dermatome map (i.e., C3 corresponds to neck pain, C4-C5 corresponds to shoulder pain, T1 corresponds to upper chest pain, L1-L2 corresponds to thigh pain, S5 corresponds to groin pain, etc.).

This database can alternatively be generated or further refined using empirical data acquired from previous patients. For example, it may be known through the stimulation treatment of previous patients that stimulating the spinal cord at the L2 spinal segment level actually provides pain relief for the right thigh, and that an active electrode combination having a cathode-anode pattern consisting of two cathodes axially separated from each other by 5 mm along the physiological midline of the spinal cord, and an anode laterally separated from these cathodes by 10 mm, and that a pulse width of 400 μs provides optimum pain relief for the right thigh. Notably, the cathode-anode pattern may, e.g., include the case electrode as the anode in a monopolar arrangement. The active electrode combination may, e.g., be expressed in terms of actual values of stimulation energy applied to the electrodes (which may be fractionalized current values) or an ideal multipole (e.g., a bipole or tripole) from which actual electrode stimulation values can be derived. In the case, where the active electrode combination is expressed in terms of actual values, these values may be fractionalized current values.

Thus, for each pain region stored within the database, a corresponding vertebral and mediolateral location, as well as other information from which a set of stimulation parameters can be derived, to optimize stimulation for the current patient is provided. In addition, “negative” information can be associated with each pain region stored in the database when side-effects (e.g., abdominal/rib stimulation, muscle stimulation, uncomfortable sensations, etc.) occur. For example, if it is known that a pulse width of >200 μs results in the side-effect of creating uncomfortable sensations in the groin region, this information may be stored in association with the pain region to be treated (in this case, the right thigh).

The processor 82 is configured for accessing the database stored in memory 84, comparing the defined therapeutic indication, which was entered by the user using the pain map 104 in the patient profile screen 100(1), to the reference therapeutic indications stored in the database, and selecting the information (including the desired stimulation target) corresponding to the reference therapeutic indication that matches the user-defined therapeutic indication. For example, if the user enters into the pain map 104 that the patient has pain in the right thigh, the information corresponding to the right thigh in the data base (including the desired stimulation target, and in this case, the midline dorsal column fibers at the L2 vertebral level) is selected.

The processor 82 is further configured for selecting the electrodes 26 adjacent the desired stimulation target based on the user-defined location of the tissue stimulation leads 12, which was entered by the user using one or more of the lead configuration screen 100(2), lead orientation screen 100(3), and drag-and-drop lead screen 100(4) described above. Because the electrodes 26 carried by the tissue stimulation leads 12 occupy different points along a two-dimensional plane, some of the electrodes 26 will be adjacent the desired stimulation target and some of the electrodes 26 will not be adjacent the desired stimulation target, assuming that at least one of the tissue stimulation leads 12 intersects the desired stimulation target.

The processor 82 may determine the electrode(s) 26 that are adjacent the desired stimulation target as potential cathodes, and if the information corresponding to the selected reference pain region includes an active electrode combination with a cathode-anode pattern, the processor 82 may select the electrodes 26 that best matches the reference cathode-anode pattern. The processor 82 may obtain the actual electrode spacing of the implanted leads 12 from, e.g., the lead type and lead number entered into patient profile screen 100(1) by the user, so that the active electrodes 26 can be best matched to the reference cathode-anode pattern. If the information corresponding to the selected reference pain region includes other reference information, such as a pulse width, the processor 82 may select that pulse width in addition to the active electrode combination. The processor 82 then generates a set of stimulation parameters, including the selected active electrode combination and pulse width. If the database has any negative information (e.g., stimulation targets, pulse widths, or cathode-anode patterns that result in side-effects), the processor 82 will takes this into account when generating the set of stimulation parameters.

In another embodiment, the processor 82 is configured for using a heuristic set of rules to generate the set of stimulation parameters based on the pain region information and lead location information input by the user.

For example, it is known that a stimulation pulse train having a relatively low pulse width (e.g., in the range of 50-300 μs) will be selective to the stimulation of dorsal roots and larger nerve fibers over dorsal columns and small nerve fibers, and a stimulation pulse train having a relatively high pulse width (e.g., in the range of 300-2000 μs) will be selective to dorsal columns and smaller nerve fibers over dorsal roots and larger nerve fibers. Based on these rules, the processor 82 may select a relatively low pulse width value as the pulse width in the generated stimulation parameter set if the user-defined pain region is of the type that is treated via stimulation of dorsal roots and/or large nerve fibers, and may select a relatively high pulse width as the pulse width in the generated stimulation parameter set if the user-defined pain region is of the type that is treated via stimulation of dorsal columns and/or small nerve fibers. Notably, the more the cathode(s) are located medially relative to the physiological midline of the spine, the more the effect of the pulsewidth on the selectivity is diminished, and the more the cathode(s) are located laterally relative to the physiological midline of the spine, the more the effect of the pulsewidth on the selectivity is amplified. These rules may also be taken into account by the processor 82 when selecting the active electrode combination.

As another example, it is known that an active electrode combination having a relatively low anode-cathode separation (e.g., less than 8 mm) will be selective to the stimulation of the midline dorsal column fibers, an active electrode combination having a relatively medial anode-cathode separation (e.g., in the range of 8-24 mm), and an active electrode combination having a relatively high anode-cathode separation (e.g., greater than 24 mm). Based on these rules, the processor 82 may select a relatively narrow anode-cathode separation for the active electrode combination if the user-defined pain region is of the type that is treated via stimulation of midline dorsal column nerve fibers, selecting a relatively medial anode-cathode separation for the active electrode combination if the user-defined pain region is of the type that is treated via stimulation of lateral dorsal column nerve fibers, and selecting a relatively wide anode-cathode separation for the active electrode combination if the user-defined pain region is of the type that is treated via stimulation of dorsal root nerve fibers.

As still another example, it is known that locating cathodes of an active electrode combination over the physiological midline of the spinal cord will be selective to the stimulation of nerve fibers from relatively more caudal dermatomes, and locating cathodes of an active electrode combination lateral to the physiological midline of the spine will be selective to the stimulation of nerve fibers from relatively more rostral dermatomes. Based on these rules, the processor 82 may select for the active electrode combination a cathode over the midline dorsal column fibers if the active electrode combination is caudal to the dorsal root fibers innervating the user-defined pain region, and may select for the active electrode combination a cathode lateral to the midline dorsal column fibers if the active electrode combination is at the same vertebral level as the dorsal root fibers innervating the user-defined pain region.

Using the generated stimulation parameter set as a starting point, the user, with the help of the CP 18, may find the optimal set of stimulation parameters to treat the pain region of the patient. Depending on the nature of the CP 18, the user may optimize the stimulation parameters in any one or more of a variety of manners using the initial set of stimulation parameters determined above. For example, the locus of the stimulation region may be electronically displaced from the locus of the stimulation region provided by the initial electrode combination by, e.g., discretely adding electrodes to and/or subtracting electrodes from the initial electrode combination, by gradually adding electrodes to and/or subtracting electrodes from the initial electrode combination to gradually shift the electrical current between the electrode array (“current steering”), by using different multiple electrode combinations for different timing channels, while adjusting the relative magnitudes of the electrical energy conveying during respective timing channels (“channel steering”).

In the case where multiple timing channels are used, the processor 82 may generate multiple electrode combinations to serve as the starting point, each one for one timing channel. Further details discussing these manners of shifting the locus of a stimulation region are described in U.S. patent application Ser. No. 11/982,704, entitled “Closed-Loop Feedback for Steering Stimulation Energy within Tissue,” which is expressly incorporated herein by reference. Furthermore, if the initial stimulation parameter set includes an active electrode combination taking the form of ideal multi-poles, the ideal poles may be displaced to displace the locus of the stimulation region, as described in U.S. Provisional Application Ser. No. 61/257,753, entitled “System and Method for Mapping Arbitrary Electric Fields to Pre-Existing Lead Electrodes,” which is expressly incorporated herein by reference. Between displacements of the locus of the stimulation region, the user may conventionally modify the pulse width or any other stimulation parameters that the user believes will provide effective therapy to the patient.

Based on patient feedback during the modification of the stimulation parameters, the user may determine one or more effective sets of stimulation parameters that have been derived from the initial set of stimulation parameters, which may then be transmitted from the CP 18 to program the IPG 14.

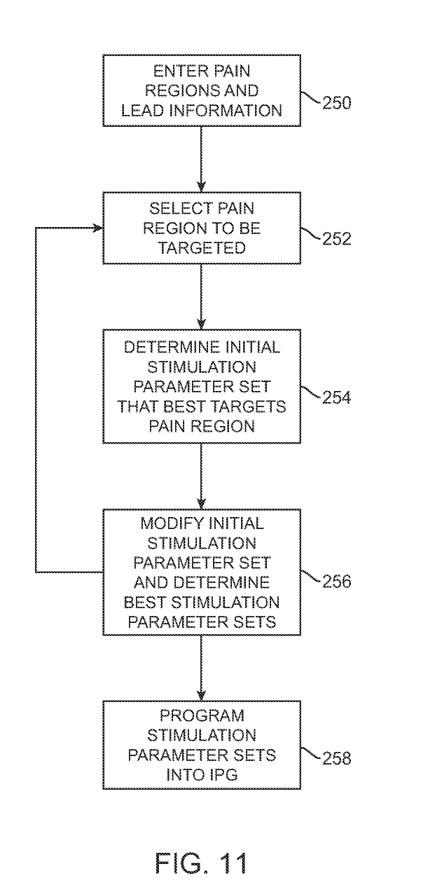

With reference to FIG. 11, in one exemplary method for programming the IPG 14, the user first enters the therapeutic indications (in this case, all of the body regions associated with pain), and the type, number, and location of the tissue stimulation lead(s) 12 relative to the anatomical structure (in this case, the spine of the patient) into the user interface of the CP 18 (step 250). The CP 18 then extracts the pain region information, and in the case, of multiple pain regions, asks the user which pain region is to be currently targeted for programming (step 252). Based on the currently targeted pain region and the lead information, the CP 18 then determines the initial set of stimulation parameters that best targets the currently targeted pain region for therapy (step 254). Starting with the initial set of stimulation parameters, the user modifies the stimulation parameters using the CP 18 and determines and saves the best few sets of stimulation parameters (if any) from the multiple ones applied based on patient feedback (step 256). Steps 250, 252, and 254 are repeated until all of the targeted pain regions have been tested and any effective stimulation parameter sets have been determined and stored, after which, the effective stimulation parameter sets are programmed into the IPG 14 (step 258).

Although the foregoing techniques have been described as being implemented in the CP 18, it should be noted that this technique may be alternatively or additionally implemented in the RC 16.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An external control device for use with a tissue stimulation device and at least one tissue stimulation lead having a plurality of electrodes implanted within a patient, comprising:
a user interface configured for allowing a user to enter first information defining a therapeutic indication and second information defining the location of the at least one tissue stimulation lead relative to an anatomical reference;
at least one processor configured for analyzing the first and second information and generating at least one set of stimulation parameters based on the analysis; and
output circuitry configured for transmitting the at least one stimulation parameter set to the tissue stimulation device.

2. The external control device of claim 1, wherein the therapeutic indication is chronic pain.

3. The external control device of claim 1, wherein the anatomical reference is the spine of the patient.

4. The external control device of claim 3, wherein location is defined relative to a vertebral level of the spine.

5. The external control device of claim 3, wherein the location is defined as a mediolateral position relative to a physiological centerline of the spine.

6. The external control device of claim 1, wherein the second information further defines a type of the at least one tissue stimulation lead.

7. The external control device of claim 1, wherein the second information further defines a number of the at least one tissue stimulation lead.

8. The external control device of claim 1, wherein the at least one tissue stimulation leads comprises a plurality of tissue stimulation leads, and the second information further defines positions of the tissue stimulation leads relative to each other.

9. The external control device of claim 1, wherein the user interface is configured for allowing the user to optimize the at least one stimulation parameter set.

10. The external control device of claim 1, further comprising memory storing a database containing a plurality of reference therapeutic indications and a plurality of desired stimulation targets respectively corresponding to the reference therapeutic indications, wherein the at least one processor is configured for analyzing the first information by comparing the defined therapeutic indication to the stored reference therapeutic indications and selecting at least one of the desired stimulation targets based on this comparison, for analyzing the second information by comparing the defined location of the at least one tissue stimulation lead to a location of the at least one desired stimulation target within the anatomical reference, and for selecting one or more of the electrodes adjacent the at least one desired stimulation target based on this comparison, wherein the at least one stimulation parameter set includes the one or more selected electrodes.

11. The external control device of claim 10, wherein the corresponding reference therapeutic indications are different pain regions.

12. The external control device of claim 10, wherein the corresponding reference therapeutic indications and desired stimulation targets are empirically determined.

13. The external control device of claim 10, wherein the database further contains a plurality of pulsewidth values respectively corresponding to the reference therapeutic indications, and the at least one processor is configured for selecting one of the pulsewidth values based on the comparison of the defined therapeutic indication to the stored reference therapeutic indications, wherein the at least one stimulation parameter set includes the selected pulsewidth value.

14. The external control device of claim 10, wherein the database further contains a plurality of anode-cathode pattern respectively corresponding to the reference therapeutic indications, and the at least one processor is configured for selecting one of the anode-cathode patterns based on the comparison of the defined therapeutic indication to the stored reference therapeutic indications, and selecting the electrodes based on the selected anode-cathode separation.

15. The external control device of claim 1, wherein the at least one processor is configured for analyzing the first information and second information using a heuristic set of rules.

16. The external control device of claim 15, wherein the at least one stimulation parameter set comprises a pulse width, and wherein the heuristic set of rules comprises selecting a relatively low pulse width value as the pulse width if the defined therapeutic indication is of the type that is treated via stimulation of dorsal roots and/or large nerve fibers, and selecting a relatively high pulse width value as the pulse width if the defined therapeutic indication is of the type that is treated via stimulation of dorsal columns and/or small nerve fibers.

17. The external control device of claim 16, wherein the relatively low pulse width value is in the range of 50-300 μs, and the relatively high pulse width value is in the range of 300-2000 μs.

18. The external control device of claim 15, wherein the at least one stimulation parameter set comprises an active electrode combination, and wherein the heuristic set of rules comprises selecting a relatively narrow anode-cathode separation for the active electrode combination if the defined therapeutic indication is of the type that is treated via stimulation of midline dorsal column nerve fibers, selecting a relatively medial anode-cathode separation for the active electrode combination if the defined therapeutic indication is of the type that is treated via stimulation of lateral dorsal column nerve fibers, and selecting a relatively wide anode-cathode separation for the active electrode combination if the defined therapeutic indication is of the type that is treated via stimulation of dorsal root nerve fibers.

19. The external control device of claim 18, wherein the relatively narrow anode-cathode separation is less than 8 mm, the relatively medial anode-cathode separation is in the range of 8 to 24 mm, and the relatively wide anode-cathode separation is greater than 24 mm.

20. The external control device of claim 15, wherein the at least one stimulation parameter set comprises an active electrode combination, and the set of heuristic rules comprises selecting for the electrode combination a cathode over the midline dorsal column fibers if the active electrode combination is caudal to the dorsal root fibers innervating a tissue region associated with the defined therapeutic indication, and selecting for the electrode combination a cathode lateral to the midline dorsal column fibers if the active electrode combination is at the same level as the dorsal root fibers innervating the tissue region associated with the defined therapeutic indication.

21. The external control device of claim 1, wherein the output circuitry comprises telemetry circuitry.

22. The external control device of claim 1, further comprising a housing containing the user interface, at least one processor, and output circuitry.
